(19) United States
(12) Patent Application Publication
(10) Pub. No.: US 2014/0271474 A1
(43) Pub. Date: Sep. 18, 2014

(54) INHIBITOR PROBES FOR IMAGING SODIUM-GLUCOSE COTRANSPORTERS IN HEALTH AND DISEASE

(71) Applicants: Ernest M. Wright, Los Angeles, CA (US); Jorge R. Barrio, Agoura Hills, CA (US)

(72) Inventors: Ernest M. Wright, Los Angeles, CA (US); Jorge R. Barrio, Agoura Hills, CA (US)

(73) Assignee: THE REGENTS OF THE UNIVERSITY OF CALIFORNIA, Oakland, CA (US)

(21) Appl. No.: 14/126,395

(22) PCT Filed: Jun. 14, 2012
(86) PCT No.: PCT/US2012/042522

§ 371 Date: May 12, 2014

Related U.S. Application Data
(60) Provisional application No. 61/497,003, filed on Jun. 14, 2011.

(51) Int. Cl. C07H 19/01; G01N 33/50; C07H 15/26; C07H 15/207; C07H 17/02

(52) U.S. Cl. CPC C07H 19/01; C07H 15/207; C07H 17/02; C07H 15/26; G01N 33/5091
USPC 424/9.1; 536/4.1; 514/35; 536/18.5; 536/18.1; 536/17.4; 514/43; 514/25; 435/29

(57) ABSTRACT

Radiolabeled tracers for binding to sodium/glucose cotransporters (SGLTs), and their synthesis, are provided. The tracers are high-affinity inhibitors of SGLTs, glycosides labeled with radioactive halogens. Also provided are in vivo and in vitro techniques for using the tracers as analytical tools to study the biodistribution and regulation of SGLTs in health and disease, and to evaluate therapeutic interventions. The ability to monitor radiolabel tracer disposition in real time enables the design of new SGLT inhibitors with lower metabolism and higher efficiency.

Benefit of U.S. Provisional Application Ser. No. 61/497,003 filed Jun. 14, 2011 is claimed.

### ACKNOWLEDGEMENT OF GOVERNMENT SUPPORT

This application was made with government support under Grant No. DK 077133, awarded by the National Institutes of Health. The Government has certain rights in the invention.

### FIELD OF THE INVENTION

The present invention relates generally to tracers and methods for detecting sodium-glucose co-transporters (SGLTs), and more particularly to radiolabeled tracers and methods for identifying and monitoring sodium/glucose cotransporters, in vitro and in viva

### BACKGROUND OF THE INVENTION

Great strides have been made over the past 30 years in the functional imaging of the human body using positron emission tomography (PET), Single-Photon Emission Computerized Tomography (SPECT), and carbon-11 and/or fluorine-18 labeled compounds. Arguably, the probe that has received the most attention is 2-deoxy-2-[18F]fluoro-D-glucose (2-FDG) and, indeed, this sustains the field of clinical PET. 2-FDG is the most widely used PET tracer in the world for in vivo assessment of regional glucose metabolic rates in humans. Approved diagnostic uses with PET include its use for detection of cancer, epilepsy, determination of myocardial viability, and Alzheimer's disease.

The success of 2-FDG PET imaging rests upon the finding that [14C]-2-deoxy-D-glucose can be used as a tracer to measure glucose metabolism in brain and other tissues. This sugar enters cells (and crosses the blood-brain-barrier) using facilitated glucose transporters (GLUTs). The glucose analog is phosphorylated by hexokinase to produce 2-deoxy-D-glucose-6-phosphate. Phosphorylated sugars are not substrates for the GLUTs, and 2-deoxy-D-glucose-6-phosphate is not further metabolized. Consequently, 2-deoxy-D-glucose-6-phosphate becomes trapped in cells. Similarly, the radiofluorinated 2-FDG is a substrate for GLUT transporters, is phosphorylated in cells to the 6-phosphate derivative, and becomes trapped.

The accumulation of 2 deoxy-2-[18F]fluoro-D-glucose- 6 phosphate(2-FDG-6P) in cells permits determination of the local rates of glucose metabolism in all tissues. Whole body-PET is employed to image 2-FDG-6P accumulation in the body. 2-FDG PET was first used to much advantage as an experimental tool to monitor regional brain activity in fully conscious subjects, and this revolutionized brain physiology. It was also found that 2-FDG was accumulated in ischemic myocardium, and FDG PET has become a tool to study cardiac pathophysiology.

For at least fifteen years, 2-FDG PET has been used to detect tumors in the body. This is based on the finding that certain tumors have a high demand for energy in the form of glucose.

### SGLTs

A second pathway for glucose entry into cells exists—the sodium/glucose cotransporter (SGLT) pathway ((Wright and Turk 2004; Wright, Hirayama et al. 2007; Wright 2010))The SGLTs use the sodium gradient across the cell membrane to “pump” sugars into cells to a level much greater than in plasma; e.g., SGLT1 pumps a specific, non-metabolized substrate (alpha-methyl-D-glucopyranoside) into cells to reach concentrations as high as 800-fold above plasma concentrations (Wright et al, 2010).

However, 2-FDG is not a substrate for these glucose transporters, and so 2-FDG PET does not measure glucose utilization into cells by the SGLTs. A hydroxyl group in the equatorial plane of the pyranose ring at carbon-2 is required for binding and transport by SGLTs (Wright et al, 2010). This means that mannose and 2-deoxy-D-glucose are poor substrates for SGLTs (Wright et al, 2010). Similarly, methyl -D-glucopyranoside (“MethylDG” or “MeDG”) is not a substrate for GLUTs (Wright et al, 2010).

There are 5 members of the family of human SLC5 gene family responsible for sodium glucose transport (SGLT1, 2, 4, 5 & 6), and one member is a glucose sensor (SGLT3): SGLT1, which is expressed mainly in the small intestine, and SGLT2, which is expressed mainly in the kidney. SGLT1 is responsible for the absorption of glucose and galactose in the human diet (180-200 grams per day), and mutations in the SGLT1 gene produce the disease Glucose-Galactose Malabsorption. SGLT2 is mainly responsible for the reabsorption of glucose from the glomerular filtrate in the kidney (180 grams/day), and mutations in this gene produce the condition known as familial renal glucosuria (FRG).

It is commonly believed that SGLT1 and SGLT2 are restricted mainly to the small intestine and kidney respectively. However, these genes are expressed throughout the body, including in the heart, lung, brain, prostate, testis, and uterus (Wright and Turk 2004; Wright 2010) and even in metastatic lesions of some tumors. Likewise, SGLT3, 4, & 6 are widely expressed throughout the body. Therefore, we believe it is reasonable to postulate that the SGLTs play a role in glucose metabolism throughout the body in health and disease (Wright 2004; Wright 2010).

Previously, we have invented a series of imaging tracers specifically to monitor glucose transport by SGLTs in health and disease, e.g. methyl-4-[18F]-4-deoxy-D-glucopyanoside (US2010/0008856A1, Wright, Barrio, Hirayama & Kepe. Tracers for monitoring the activity of sodium glucose/cotransporters in Health and Disease). It has been established that in healthy subjects SGLTs are active throughout the body in addition to the small intestine and kidney, including brain, heart skeletal muscle, prostate gland, testis and ovary. In addition, in humans, SGLT imaging probes are useful in detecting cancer, e.g. prostate and brain, and identifying deficits in SGLT activity in Friedrich's ataxia.

### Diabetes

This chronic disease is a disorder of glucose homeostasis where blood glucose levels greatly exceed the normal levels, >>10 mM. If hyperglycemia is left untreated it results in glucose toxicity, which damages blood vessels, and peripheral nerves leading to blindness, kidney failure, peripheral neuropathy, cardiovascular disease, and other serious complications. It is estimated that 25 million patients in the US have diabetes, and the number is growing. One of the earliest symptoms is a loss of glucose to the urine due to hyperglycemia overwhelming the reabsorption capacity of SGLTs in the proximal tubule. Current therapies to combat this disease are centered on controlling blood glucose levels by increasing insulin secretion, improving insulin sensitivity, and reducing liver glucose output and intestinal glucose absorption. As the disease progresses, patients require combinations of medicines and, unfortunately, adverse side effects compromise compliance and the health of the patient.

There are growing efforts in finding alternative therapies to manage diabetic patients, and one is to control blood glucose by using inhibitors to reduce intestinal glucose absorption and renal reabsorption by inhibiting SGLT1 in the intestine and SGLT2 in the kidney (Chao and Henry 2010). The proof of concept for SGLT2 targeted therapy was provided by Oku and colleagues (Oku, Ueta et al. 1999) who demonstrated that a pro-drug, T-1095, was absorbed from the gut into the circulation; this resulted in renal glucose excretion in diabetic animals and lowered blood glucose levels. T1095 also suppressed post-prandial hyperglycemia and reduced hyperinsulinemia and hypertriglyceridemia in diabetic rodents. In the decade since there have been at least 21 SGLT2 inhibitors that entered the drug pipeline. Most have exploited the same chemical space as phlorizin (see FIG. 1), and many of these compounds are in Phase I to III clinical trials (Table 1). FIG. 1 shows the chemical formulas for several SGLT. Phlorizin and T-1095 are nonselective for the sodium-glucose co-transporters (SGLTs), whereas sergliflozin and remogliflozin exhibit markedly increased selectivity for SGLT2.

Agents currently in development. All agents are selective for SGLT2, except for DSP-3235, which targets SGLT1 (Chao and Henry 2010)). To cite one example, we focus on the C-aryl glucoside dapagliflozin (FIG. 1). This drug had a SGLT2 inhibitor constant (EC₅₀) of 1 nM and a selectivity of 1,200 for SGLT2 over SGLT1(Washburn 2009), which make its radiolabeled derivatives ideal for imaging.

TABLE 1

| Drug | Company | Status |
|---|---|---|
| Dapagliflozin | Bristol-Myers Squibb/ | Phase III |
| (BMS-512148) | AstraZeneca | |
| Canagliflozin | Johnson & Johnson/ | Phase III |
| (TA-7284, | Mitsubishi Tanabe Pharma | |
| JNJ-28431754) | | |
| ASP-1941 | Astellas/Kotobuki | Phase III |
| BI-10773 | Boehringer Ingelheim | Phase II |
| BI-44847 | Boehringer Ingelheim (under | Phase II |
| | license from Ajinomoto) | |
| TS-071 | Taisho Pharmaceutical | Phase II |
| CSG-452 | Roche/Chugai Pharmaceutical | Phase II |
| (R-7201, | | |
| RG-7201) | | |
| LX-4211 | Lexicon Pharmaceuticals | Phase II |
| DSP-3235 | GlaxoSmithKline/Dainippon | Phase I |
| (KGA-3235, | Sumitomo (under | |
| GSK-1614235, | license from Kissei | |
| 1614235)* | Pharmaceuticals) | |

*Selective for SGLT1; all other agents are selective for SGLT2. (Chao & Henry, 2010 (Chao and Henry 2010)).

Phase II clinical trials with type 2 diabetic patients for 242 weeks have been published (Komoroski, Vachharajani et al. 2009) (List, Woo et al. 2009) and Phase III trials for up to 48 weeks have been reported in abstract form. In general, the tested doses of dapagliflozin produce a sustained 30-65 gram/day urinary glucose excretion, a 22±10% reduction in fasting serum glucose, and a 20±10% reduction in post-prandial glucose absorption (area under the plasma glucose concentration curve).The loss of urinary glucose, 200-300 kcal/day results in weight loss (up to 3.5 Kg over 28-58 weeks), increases in urine volume (up to 470 ml/day) and hematocrit (up to 3%), and an associated modest reduction in diastolic blood pressure of 2-5 mm Hg. The results so far suggest that anti-SGLT2 inhibitors may be useful in reaching the goals for low glycemic control in type 2 diabetic patients and reducing glycosylated hemoglobin (Hb A1C) levels to less than 7%. According to these reports, the dapagliflozin treatment for up to 48 weeks produces no remarkable clinical side effects relative to the placebo controls, as expected from the long term follow up with one patient with massive Familial Renal Glucosuria (Scholl-Burgi, Santer et al. 2004).

In summary, the pharmaceutical industry has advanced the use of SGLT1 and SGLT2 inhibitors for managing hyperglycemia in diabetic patients from Phase I to Phase III clinical trials. The FDA has accepted an application to use a SGLT2 inhibitor from one company (December 2010). Others in the industry have developed inhibitors to control blood glucose in diabetic patients specific by reducing intestinal glucose absorption, i.e. SGLT1 specific inhibitors (see Table 1 and FIG. 1). A major issue that arises with the development of new drugs, including the SGLT inhibitors in this class, is their metabolism in the intestine and liver in human subjects and patients. In the case of dapagliflozin the major metabolite, dapagliflozin 3-O-glucuronide, is reported to be inactive ((Kasichayanula, Chang et al.; Obermeier, Yao et al. 2010). After oral dosing with dapagliflozin the plasma level of the metabolite is higher than the native drug for 1-25 hours after administration.

### Cancer and Glucose

Glucose is a major source of energy and the demand for glucose in cancer cells is even higher than in normal cells. This is the basis for the detection and staging of tumors using 2FDG. However, some tumors do not accumulate 2-FDG, a substrate for GLUTs but not SGLTs, increasing interest in the expression of SGLTs in cancer. Inspection of the EST data bases, which can be located at www.ncbi.nlm.nih.gov/unigene, indicates that SGLT1 is expressed in colorectal, head and neck, and prostate tumors, and SGLT2 is expressed in colorectal, GI, head and neck, kidney tumors, chondrosarcomas and in leukemia. There are a handful of publications on the mRNA levels of SGLT1 and SGLT2 and immunohistochemistry of SGLT1 in primary tumors, and metastatic lesions of lung, pancreatic adenocarcinomas, and head and neck cancers (Ishikawa, Oguri et al. 2001; Helmke, Reisser et al. 2004). SGLT1 was expressed in well differentiated squamous cultures of head and neck carcinomas; SGLT2 was expressed in metastatic lesions of lung cancers; and SGLT1 protein was reported to be expressed in primary pancreatic adenocarcinomas. Using Me-4-[18F]fluoro-4-deoxy-D-glucopyranoside as a probe we also have confirmed in living human subjects expression of SGLTs in prostate cancer and glioblastomas. Other anticipated uses of the specific SGLT inhibitors are to reduce the growth of tumors expressing SGLT2s (WO 2009117367) and SGLT1.

### SUMMARY

We have discovered that certain radiolabeled SGLT inhibitors are remarkably well-suited for use as radiographic tracers for sodium/glucose cotransporters, both in vitro and in vivo. According to one aspect of the invention, a tracer for an inhibitor of SGLT2 is provided comprising, for example, a glucopyranoside radiolabeled with 18F, 123I, or 124I, as is described below in more detail. An exemplary tracer is the radiofluorinated compound, [18F]-dapagliflozin ((1S)-1,5-anhydro-1-C-{4-chloro-3-[(4-ethoxyphenyl)methyl]phenyl}-4-[18F]-4-deoxy-D-glucitol or [18F]-Dapa).

[chemical structure]

It is a specific, high-affinity inhibitor (Kᵢ 1- 5 nanomolar) for SGLT2. A method of making radiolabeled inhibitors pyranosides is also provided.

In a second aspect of the invention, a method of detecting SGLTs in vitro is provided. Radiographic techniques include, without limitation autoradiography. Introduction of a fluorophore on the probe permits its use in optical methods. This method can be enhanced by using it to monitor the effect on the cellular sample of one or more administered pharmacological or other agents.

In a third aspect of the invention, a method of assessing sodium/glucose cotransporter distribution in a human or non-human mammal, in vivo, is provided, by administering to the mammal a bolus of a tracer and measuring the time-dependent distribution of activity in the mammal body generating radiographic data indicative of tracer uptake in the specific tissue target (SGLT) by scanning the mammal using a radiographic technique (or optimal imaging method); and using the radiographic data to assess SGLT distribution in the mammal (FIG. 2). FIG. 2 comprises microPET images of a rat injected with [18F]-dapagliflozin where (a) shows [18F]-dapagliflozin binding to the outer cortex of the kidney where SGLT2 is expressed. With pre-injection of dapagliflozin as shown in (b) or phlorizin (Pz) as shown in (c.), [18F]-dapagliflozin binding to the kidney cortex was completely blocked. The images were summed from 50-60 minutes post injection [18H-dapagliflozin. In b) and c) note the [18F-activity in the intestine is due to excretion by the liver into bile.

In a variation of this method, a tracer that is known to be an inhibitor for SGLT2, but not SGLT1 (or vice versa), is utilized, allowing one to determine the distribution and pharmacokinetics of any unlabeled inhibitor (e.g., experimental drug). This is illustrated for dapagliflozin in FIG. 2C. The techniques described herein permit comparative studies of drug binding to SGLTs (FIG. 2) and the study of other pharmacological or other agents on the level of SGLT expression in any given tissue, e.g. insulin, to better assess the agent's usefulness (and/or its deleterious effect) on the mammal.

Thus, it is possible in real time to follow the binding of the inhibitors or drug candidates to SGLTs in target and off-target organs in the body, e.g. [18F]-Dapa binding to SGLT2 kidney cortex, and other tissues expressing SGLT2, e.g. heart, brain, testis and ovary.

It is also possible to compare the potency of SGLT inhibitors, e.g. quantitate the effect of drug doses on the pharmacokinetics of a SGLT2 radiolabeled probe, for example, displacement studies of DAPA, Canaglifozin, LX-4211, and BI-10773 on [18F]-Dapa or other [18F]-inhibitors. Similarly, the site of action of drugs beyond the target tissue can be also evaluated in animals and humans, permitting a direct method to establish phamacological efficacy (on target tissue; e.g., kidney) and adverse effects (e.g., cardiovascular action) and their correlation with drug doses. Applicants conducted preliminary studies of this action in both non-human mammals and human subjects.

A fourth aspect of the invention is the identification of tumor cells expressing a specific SGLT isoform, e.g. SGLT2, allowing the selection of specific SGLT inhibitors for inhibiting glucose uptake into tumors to block their growth, and monitoring the effectiveness of the therapy.

A fifth aspect of the invention is to follow, in real time, the metabolism and elimination of SGLT drug metabolites by the liver and/or kidney (e.g., by using a specific radiolabeled drug). This permits the exploitation of methods to reduce drug metabolism and increase efficacy, e.g. reduce glucuronidation of SGLT2 inhibitors by pharmacological methods and/or chemical modification of the SGLT2 inhibitor.

A sixth aspect of the invention is the identification of new improved SGLT2 inhibitors with low rates of glucuronidation in vivo. This reduces the rapid metabolism of the inhibitors in human subjects, increases the life time of the parent drug, and reduces the potential for adverse reactions of the metabolites.

### BRIEF DESCRIPTION OF DRAWINGS

FIG. 1, presented as FIGS. 1aand 1b for clarity, shows the chemical formulas for several prior art sodium-glucose co-transporters inhibitors.

FIG. 2 comprises three radiographic images illustrating the time dependent distribution of tracer uptake in specific tissue targets.

FIG. 3 is a schematic representation of multiple radiolabeled compounds for SGLT.

FIG. 4 shows a chemical reaction scheme for the synthesis of galacto-Dapa triflate phenol.

FIG. 5 illustrates a procedure for preparing [18F] dapaglifozin from galacto-DAPA triflate.

FIG. 6 illustrates a procedure for preparing phenol-aglycon.

FIG. 7 illustrates a generalized reaction scheme using various aryl compounds, corresponding to the specific reactions of FIGS. 4 and 5, with gluconolactone as a starting sugar.

### DETAILED DISCUSSION

The first aspect of the invention is the synthesis of [18F]fluoro, and [123I]iodo- SGLT inhibitors.

FIG. 3 provides a schematic description of radiolabeled tracers for SGLTs incorporating features of the invention. These radio labeled tracers comprise a sugar moiety (the 6 membered ring shown on the left of FIG. 3) connected by Z to ring A which is in turn connected to ring B by Y, one or more of the substitutions in the sugar moiety, ring A or ring B being a radiolabeled halogen. Ring A and B are the same or different phenyl, heterocyclic or fused aromatic rings.

Representative examples of radiolabeled SGLT inhibitors within the scope of FIG. 3 include, for example, the following compounds:

TABLE 3

| | |
|---|---|
| 4-[¹⁸F]-phlorizin (¹⁸F on C4 of D-glucose) | [chemical structure] |
| [¹⁸F]-phlorizin (¹⁸F on B-ring of aglycone) | [chemical structure] |
| 4-[¹⁸F]-dapagliflozin (¹⁸F on C4 of D-glucose) | [chemical structure] |
| 3-[¹⁸F]-dapagliflozin (¹⁸F on C3 of D-glucose) | [chemical structure] |
| 4-[¹⁸F]-3-O—Me-dapagliflozin (¹⁸F on C4 of 3-O—Me-D-glucose) | [chemical structure] |
| [¹⁸F]-3-O—Me-dapagliflozin (¹⁸F on A-ring of aglycone) | [chemical structure] |
| 4-[¹⁸F]-canagliflozin (¹⁸F on C4 of D-glucose) | [chemical structure] |
| [¹⁸F]-canagliflozin (¹⁸F on B-ring of aglycone) | [chemical structure] |
| 3-[¹⁸F]-LX-4211 (¹⁸F on C3 of L-xylose) | [chemical structure] |
| 4-[¹⁸F]-DSP-3235 (¹⁸F on C4 of D-glucose) | [chemical structure] |
| 3-[¹⁸F]-TS-071 (¹⁸F on C3 of thio-D-glucose) | [chemical structure] |
| 4-[¹⁸F]-CSG452 (a.k.a. “tofogliflozin”) (¹⁸F on C4 of D-glucose) | [chemical structure] |
| 4-[¹⁸F]-BI-10 (¹⁸F on C4 of D-glucose) | [chemical structure] |
| [¹⁸F]-BI-10 (¹⁸F on A-ring of aglycone) | [chemical structure] |

An example describing the synthesis of one embodiment of the novel labeled tracers as set forth in FIG. 3, and their use in vivo and in vitro, is also provided. Synthesizing other radiolabeled tracers listed in FIG. 3 is straightforward based on the teachings herein. For example, according to a first aspect of the invention, such a tracer comprises a radiolabeled SGLT1 or SGLT2 inhibitor, e.g. the SGLT2 inhibitor [18F]-dapagliflozin; [18F]DAPA; 18F-ECFP) ((1S)-1,5-anhydro-1-C-{4-chloro-3-[(4-ethoxyphenyl)methyl]phenyl}-4-[18F]-4-deoxy-D-glucitol) whose synthesis is depicted in FIGS. 4 and 5. FIGS. 6 and 7 are a generalized example of a reaction scheme using various aryl compounds, corresponding to the specific reactions of FIGS. 4 and 5 with gluconolactone as a starting sugar in FIG. 7 (such as more generally shown as a component of the compound of FIG. 3). This generalized example is applicable to the preparation of the various radiolabeled SGLT inhibitors disclosed herein. It should be recognized that the gluconolactone can be replaced, in FIG. 7, by other sugars as a starting material.

In regard to the sixth aspect set forth above it has been discovered that new and improved SGLT2 inhibitors included within the composition of FIG. 3 which have 3-O-alkyl-, particularly 3-O-methyl, or 3-deoxy-D-glucose derivative have a low in vivo metabolism when use for the treatment of diabetes. Such compounds eliminate or reduce glucuronidation of active SGLT2 inhibitors, such as 3-O-methyl-4-[F]-4-deoxy-Dapagliflozin, and thereby improve efficiency and plasma lifetime of the active drug in vivo.

The following sequential steps are taken for the preparation of [18F]dapagliflozin ([18F]DAPA; 18F-ECFP):
a. The backbone SGLT inhibitor structure, Dapagliflozin, is synthesized as shown in the synthesis scheme of FIG. 4, by reacting gluconolactone with a phenol-aglycon (see FIG. 6 for phenol-aglycon preparation) by following the procedure set forth in J. Med. Chem. 2008; 51:1145-1149, incorporated herein in its entirety by reference. (Meng W, Ellsworth B A, Nirschl A A, McCann P J, Patel M, Girotra R N, Wu G, Sher P M, Morrison E P, Biller S A, Zahler R, Deshpande P P, Pullockaran A, Hagan D L, Morgan N, Taylor J R, Obermeier M T, Humphreys W G, Khanna A, Discenza L, Robertson J G, Wang A, Han S Wetterau J R, Janovitz E B, Flint O P, Whaley J M, Washburn W N (2008). Discovery of dapagliflozin: a potent, selective renal sodium-dependent glucose cotransporter 2 (SGLT2) inhibitor for the treatment of type 2 diabetes. J Med. Chem 51:1145-1149, 2008) The phenol-aglycon was prepared from the bromo-aglycon by lithiation and subsequent electrophilic substitution reaction (FIG. 4 and more generally shown in FIG. 6). The bromo-aglycon itself was synthesized by following the procedure reported in J. Med. Chem. 2008; 51:1145-1149 incorporated herein in its entirety (See above). Typical spectrometric characteristics of the phenol-aglycon and dapaglifloxin are indicated below:

Phenol-aglycon. Negative-mode ESI MS: Calcd for C₁₅H₁₅ClO₂. 262.08; Found 261.2 (M−1). ¹H NMR (300 MHz, Chloroform-d): δ 7.23 (d, J=8.6 Hz, 1H), 7.12 (d, J=8.7 Hz, 2H), 6.85 (d, J=8.7 Hz, 2H), 6.65 (dd, J=8.6 and 3.0 Hz, 1H), 6.57 (d, J=3.0 Hz, 1H), 4.91 (s, 1H), 4.03 (q, J=7.0 Hz, 2H), 3.98 (s, 2H), 1.42 (t, J=7.0 Hz, 3H) ppm. ¹³C NMR (75 MHz, Chloroform-d) δ 157.3, 154.1, 140.3, 131.0, 130.1, 129.9, 125.3, 117.4, 114.4, 63.3, 38.2, 14.7 ppm.

Dapagliflozin. ESI MS: Calcd for C₂₁H₂₄ClFO₅ 410.13; Found 433.10 (M+Na). ¹H NMR (300 MHz, chloroform-d): δ 7.34 (d, J=8.6 Hz, 1H), 7.17-7.12 (m, 2H), 7.08 (d, J=8.50 Hz, 1H), 6.80 (d, J=8.50 Hz, 1H), 4.40 (dt, J=50.8 and 9.1 Hz, 1H), 3.89-4.14 (m, 5H), 3.60-3.88 (m, 3H), 3.48 (m, 1H), 3.39 (t, J=9.2 Hz, 1H), 1.38 (t, J=7.0 Hz, 3H) ppm. ¹³C NMR (75 MHz, chloroform-d) δ 157.4, 139.3, 136.5, 134.4, 131.1, 130.4, 129.8, 129.8, 126.4, 114.5, 88.9 0 =181.3 Hz), 81.0, 77.5(J=22.6 Hz), 76.1 (J=16.9 Hz), 74.5 (J=8.5 Hz), 63.5, 61.3, 38.3, 14.9 ppm. ¹⁹F NMR (282 MHz, chloroform-d) δ-198.7 ppm.
b. The synthesis of the precursor material for radiofluorination to produce 18F-DAPA, namely galacto-Dapa triflate, is described in FIG. 4, which also describes the synthesis of the authentic, unlabeled 4-fluorodapaglifozin (FDAPA or ECFP). β-Dapa was converted to the galactopyranoside 4 via the reaction with sodium nitrite. The galactopyranoside 4 was then either fluorinated with N,N-dimethylaminosulfuryltrifluoride (DAST) followed by deprotection to give the 4′-fluoroglucopyranoside ECFP (or FDAPA) or reacted with trifluoromethlenesulfonyl anhydride (Tfl₂O) (i.e triflation) to give the galacto-DAPA triflate precursor (ECFP-Tfl). This triflate precursor is used for radiofluorination to produce 18F-DAPA as described in FIG. 5. The pure precursor ECFP-Tfl was obtained by chromatography on a silica gel column with dichloromethane/MeOH (99.5:0.5) as eluent followed by recrystallization from ethyl acetate/hexane. Detailed synthesis procedures are described below:

### Typical Synthetic Procedure for ¹⁸F-Dapa Precursor

2′,3′,6′-Tri-O-acetyl-Dapa (2). A mixture of β-Dapa (1.37 g, 3.36 mmol), tBuMe₂SiCl (653 mg, 4.35 mmol) and imidazole (455 mg, 6.7 mmol) in anhydrous (50 mL) was stirred at room temperature for 18 hours. The reaction was quenched with water (20 mL). The separated organic phase was dried with NaSO₄, filtered and evaporated. The gel-like residue was co-evaporated with anhydrous pyridine (2×5 mL) and then re-dissolved in anhydrous pyridine (20 mL). To the pyridine solution was added Ac₂O (5 mL). The reaction was stirred at room temperature for 24 hr and then quenched with MeOH (5 mL) at 0° C. The mixture was evaporated and co-evaporated with toluene (3×10 mL), giving crude product 1 (2.54 g). The crude product 1 was dissolved in 80% HOAc (60 mL) (Milton J, J. Med. Chem. 1996; 39, 1314-1320) and stirred at 50° C. for 24 hr. The reaction mixture was evaporated and co-evaporated with anhydrous toluene (2×10 mL) and anhydrous acetonitrile (2×10 mL). The dried residue was reacted again with tBuMe₂SiCl (450 mg, 3 mmol) and imidazole (315 mg, 4.58 mmol) in anhydrous dichloromethane (35 mL) at room temperature for 18 hr. After worked up as above, the crude product was applied to a silica gel (60 g) column for chromatography. The column was eluted with dichloromethane/hexane (8:2) followed by dichloromethane/EtOAc (9:1) and dichloromethane/MeOH (97:3), giving product 2 (355 mg). Recovered product 1 (1.19 g) was re-used for reaction with 80% HOAc to produce 2 [symbol] ,3 [symbol] ,6 [symbol] -tri-O-acetyl-Dapa (2) in total yields of 75-80%.

6′-O-Tert-Butyldimethylsilyl-2′,3′,4′-tri-O-Acetyl-Dapa (1), ¹H NMR (CDCl₃, 360 MHz): δ 7.35 (d, J=10.0 Hz, 1H), 7.19 (dd, J=10.0 and 2.1 Hz, 1H), 7.11 (d, J=2.1 Hz), 7.06 (d, J=10.4 Hz, 2H), 6.82 (d, J=10.4 Hz, 2H), 5.28 (d, J=10.9 Hz, 1H), 5.22 (t, J=11.3 Hz, 1H), 5.01 (t, J=11.3 Hz, 1H), 4.30 (d, J=11.7 Hz, 1H), 4.10-3.94 (m, 4H), 3.77 (dd, J=13.7 and 2.5 Hz, 1H), 3.72 (dd, J=13.7 and 4.5 Hz, 1H), 3.64 (m, 1H), 2.05 (s, 3H), 2.00 (s, 3H), 1.73 (s, 3H), 1.41 (t, J=7.0 Hz, 3H), 0.87 (s, 9H) ppm. ¹³C NMR (CDCl₃, 75.5 MHz): δ 170.57, 169.39, 168.85, 157.44, 138.95, 135.63, 134.35, 131.13, 129.79, 129.62, 126.08, 114.45, 79.36, 78.98, 74.50, 72.90, 68.85, 63.35, 62.49, 38.30, 25.79, 20.75, 20.73, 20.36, 18.32, 14.88 ppm.

2′,3′,6′-Tri-O-Acetyl-Dapa (2), ¹H NMR (CDCl₃, 360 MHz): δ 7.35 (d, J=10.1 Hz, 1H), 7.19 (dd, J=10.1 and 1.8 Hz, 1H), 7.09-7.04 (m, 3H), 6.82 (d, J=10.3 Hz, 2H), 5.15 (t, J=10.7 Hz, 1H), 4.97 (t, J=11.8 Hz, 1H), 4.44 (dd, J=14.9 and 3.7 Hz, 1H), 4.37 (dd, J=14.9 and 1.6 Hz, 1H), 4.32 (d, J=11.8 HZ, 1H), 4.09-3.94 (m, 4H), 3.75-3.62 (m, 2H), 3.44 (d, J=4.7 Hz, 1H), 2.11 (s, 3H), 2.01 (s, 3H), 1.72 (s, 3H), 1.40 (t, J=7.0 Hz, 3H) ppm. ¹³C NMR (CDCl₃, 75.5 MHz): δ 171.65, 171.24, 169.00, 157.30, 138.82, 135.40, 134.27, 130.96, 129.72, 129.68, 129.61, 125 88, 114.32, 79.17, 78.14, 76.46, 72.42, 68.80, 63.23, 63.17, 38.11, 20.75, 20.23, 14.73 ppm. ESI MS: Calcd 534.17 (C₂₇H₃₁ClO₉); Found 557.20 (M+Na).

2′,3′,6′-Tri-O-acetyl-4′-O-triflyl-Dapa (3). To the solution of compound 2 (430 mg, 0.75 mmol) in anhydrous pyridine (6 mL) at −20° C. was added triflyl anhydride (0.38 mL, 2.25 mmol). The reaction mixture was stirred at room temperature for 2.5 hr and then quenched with MeOH (0.2 mL) at −20° C. After evaporation and co-evaporation with toluene (2×3 mL), the product was purified by silica gel (25 g) chromatography using a dicloromethane/EtOAc gradient (0-5% EtOAc), giving compound 3 (385 mg, 77% yield). ¹H NMR (CDCl₃, 360 MHz): δ 7.38 (d, J=10.0 Hz, 1H), 7.16 (dd, J=10.0 and 2.1 Hz, 1H), 7.09-7.04 (m, 3H), 6.84 (d, J=10.4 Hz, 2H), 5.48 (t, J=11.4 Hz, 1H), 5.18 (t, J=11.7 Hz, 1H), 5.05 (t, J=11.7 Hz, 1H), 4.40 (dd, J=15.2 and 2.5 Hz, 1H), 4.39 (d, J=11.7 Hz, 1H), 4.25 (dd, J=15.2 and 3.7 Hz, 1H), 4.11-3.92 (m, 5H), 2.13 (s, 3H), 2.09 (s, 3H), 1.75 (s, 3H), 1.42 (t, J=7.0 Hz, 3H) ppm. ¹³C NMR (CDCl₃, 75.5 MHz): δ 170.29, 169.84, 168.84, 157.56, 139.37, 134.93, 134.24, 130.87, 129.91, 129.85, 129.63, 125.77, 114.53, 79.63, 78.89, 75.13, 72.72, 63.40, 63.17, 61.71, 38.24, 20.56, 20.48, 20.22, 14.88 ppm. ¹⁹F NMR (CDCl3, 282.4 MHz): δ −74.58 ppm. ESI MS: Calcd 666.11 (C₂₈H₃₀ClF3O₁₁S); Found 688.90. (M+Na).

2′,3′,6′-Tri-O-actyl-galacto-Dapa (4). To the solution of 3 (375 mg, 0.56 mmol) in anhydrous DMF (0.8 mL) was added NaNO₂ (310 mg, 4.5 mmol). The reaction mixture was stirred at room temperature for 7 hr. The reaction mixture was evaporated and the product purified by silica gel (20 g) chromatography, using a dichloromethane/EtOAc gradient (0-10%), giving compound 4 (152 mg, 50% yield). ¹H NMR (CDCl₃, 360 MHz): δ 7.36 (d, J=9.9 Hz, 1H), 7.26 (dd, J=9.9 and 2.1 Hz, 1H), 7.12 (d, J=2.1 Hz, 1H), 7.07 (d, J=10.2 Hz, 2H), 6.82 (d, J=10.2 Hz, 2H), 5.36 (t, J=11.8 Hz, 1H), 5.09 (dd, J=11.8 and 3.1Hz, 1H), 4.40 (dd, J=14.0 and 6.1 Hz, 1H), 4.29 (dd, J=14.0 and 6.1 Hz, 1H), 4.26 (d, J=11.8 Hz, 1H), 4.15 (t, J=3.1 Hz, 1H), 4.10-3.94 (m, 4H), 3.87 (t, J=6.1 Hz, 1H), 2.61 (d, J=4.0 Hz, 1H), 2.10 (s, 3H), 2.08 (s, 3H), 1.73 (s, 3H), 1.41 (t, J=7.0 Hz, 3H) ppm. ¹³C NMR (CDCl₃, 75.5 MHz): δ 171.09, 170.23, 169.14, 157.43, 138.83, 135.71, 134.43, 131.20, 130.09, 129.81, 129.79, 126.27, 114.46, 79.94, 75.96, 74.31, 70.16, 67.72, 63.38, 62.87, 38.27, 20.91, 20.43, 14.88 ppm. ESI MS: Calcd 534.17 (C₂₇H₃₁ClO₉); Found 557.27 (M+Na).

Galacto-Dapa triflate. To the solution of compound 4 (367.6 mg, 0.688 mmol) in anhydrous pyridine (3.5 mL) at −20° C. was added triflyl anhydride (0.175 mL, 1 mmol) dropwise. The reaction mixture was stirred and gradually allowed to reach room temperature and maintained at room temperature for 2 hr. The reaction was quenched with MeOH (0.2 mL) at −20° C. After evaporation and co-evaporation with toluene (2×3 mL), the product was purified by silica gel (20 g) chromatography using dichloromethane and dichloromethane/MeOH (99.5:0.5), giving galacto-Dapa triflate (331 mg, 72.2% yield). ¹H NMR (CDCl₃, 360 MHz): δ 7.40 (d, J=10.0 Hz, 1H), 7.23 (dd, J=10.0 and 1.9 Hz, 1H), 7.08 (d, J=10.4 Hz, 2H), 7.05 (d, J=1.9 Hz, 1H), 6.84 (d, J=10.4 Hz, 2H), 5.36 (d, J=2.8 Hz, 1H), 5.29 (d, J=11.3 Hz, 1H), 5.23 (dd, J=12.2 and 3.0 Hz, 1H), 4.40-4.31 (m, 2H), 4.15-3.96 (m, 6H), 2.13 (s, 3H), 2.09 (s, 3H), 1.74 (s, 3H), 1.42 (t, J=7.0 Hz, 3H) ppm. ¹³C NMR (CDCl₃, 75.5 MHz): δ 170.17, 168.37, 157.53, 139.17, 134.92, 134.48, 130.93, 129.96, 129.85, 126.07, 114.50, 81.56, 80.49, 74.05, 70.90, 68.95, 63.38, 61.00, 38.22, 20.59, 20.29, 14.87 ppm. ¹⁹F NMR (CDCl3, 282.4 MHz): δ −74.05 ppm. ESI MS: Calcd 666.11 (C₂₈H₃₀ClF3O₁₁S); Found 689.00. (M+Na).
c. [18F] dapaglifozin (the half-life of [18]-fluorine is 109 minutes) was prepared from the galacto-Dapa triflate by a common standard nucleophlic [18F] labeling procedure using [18F]fluoride either in solution or having the [18F]fluoride adsorbed on solid surfaces. The radiosynthesis scheme is depicted in FIG. 5. The specific radioactivity was >4,000 Ci/mmol at end of synthesis: the half-life of the 18F] isotope is 109 minutes. The structure of the all unlabeled products was confirmed by ESI MS, 1H NMR 13C NMR, 19F NMR, radioTLC, HPLC, and X-ray crystallography. RadioTLC was used for characterization of radiolabeled materials using authentic samples as reference. A detailed procedure is described below:

[18F1-dapagliflozin ([18F]-Dapa or [18F]-ECFP). [¹⁸F]Fluoride was made in a Cyclotron by proton bombardment on ¹⁸O enriched water via the ¹⁸O(p,n)¹⁸F nuclear reaction. Galacto-Dapa triflate (DAPA-Tfl) (5 mg) in anhydrous acetonitrile (0.5 mL) was added to a dried ¹⁸F-ion/K₂CO₃ (1 mg)/Kryptofix (10 mg) residue following standard procedures and reacted at 90° C. for 15 min. The ¹⁸F-ion can also be adsorbed on a solid surface material with similar results. The reaction mixture was diluted with water (3 mL) and pre-purified with a C18 Sep-Pak cartridge. The product extracted on the Sep-Pak was washed with water (2×4 mL) and eluted off with MeOH (1.5 mL). To the MeOH solution was added LiOH (1M, 0.4 mL). After neutralization in 5 min with HCl (2 M, 0.2 mL), the resulted mixture was injected to the semi-prep HPLC (Grace Altima C18, 5μ, 10×150 mm; 42% EtOH in water, 4 mL/min) for purification. The collected product fraction (RT=24 min) was diluted with an equal volume of water and extracted with a C-18 Sep-Pak cartridge. After wash with sterile water (20 mL), the product was eluted from the cartridge with absolute EtOH (0.3-0.5 mL), giving [18F]-Dapa in 38% decay corrected radiochemical yields. The EtOH solution of [18F]-Dapa was diluted with 9 volume of saline and filtered through sterile filter for injection. Typical reactions produce 80 mCi of product from 420 mCi of (18F)fluoride after about 2-hour synthesis and processing time to produce sterile and pyrogen-free injectable solution of the radiopharmaceutical. Proportionally higher mCi amounts of [18F]-Dapa can be easily produced starting from multiCi amounts of (18F)fluoride, which is easily produced in most current medical cyclotrons (e.g., 11 MeV or larger). Typically, 10-mCi of [18F]-Dapa (18F-ECFP) are used for each human PET scan.

QC: RadioTLC (C18; THF/MeOH/water 4:4:2); HPLC (Waters Symmetry C18, 5 m, 4.6×150 mm; MeCN/water 4:6, 1 mL/min, 254 nm); and pyrogenicity tests. Radioactive purity: >99%; Chemical purity >90%; Specific radioactivity >4000 Ci/mmol at end of synthesis. The pure authentic sample of F-Dapa (ECFP) (FIG. 4) was obtained by chromatography on silica gel column with dichloromethane/methanol (95:5) as eluent.

In a second aspect of the invention, a method of detecting a sodium/glucose co-transporter in vitro is provided, and comprises the steps of obtaining a cellular sample; administering to the cellular sample a radiolabeled inhibitor as described herein (e.g., a tracer listed in FIG. 3); isolating a first aliquot of the cellular sample after a first time interval and washing it with a buffer solution; assaying the first aliquot for radioactivity; and, after each of one or more additional time intervals, isolating a further aliquot of the cellular sample, washing it with a buffer solution, and assaying it for radioactivity. Radiographic techniques include, but are not limited to, autoradiography. This method can be enhanced by using it to monitor the effect on the cellular sample of one or more administered pharmacological or other agents.

Examples of pharmacological agents include, but are not limited to, sodium ion, glucose, galactose, phlorizin, SGLT inhibitors, and insulin. A reference describing the methods for assaying radioactive uptake and binding in isolated tissues and cells is Hummel et al (2011)(Hummel 2010).

In a third aspect of the invention, in vivo methods of monitoring SGLTs in mammals, whether healthy or diseased, are provided. In one embodiment, a bolus of a radiolabeled tracer is administered to a mammal; radiographic data indicative of tracer uptake is generated by scanning the mammal using a radiographic technique; and the radiographic data that is generated is used to probe or assess SGLT distribution or activity in the mammal. One variant of this aspect is to determine the oral bioavailability of the SGLT inhibitors by administration of the bolus of radiolabeled tracer by mouth.

This aspect of the invention can be used to monitor SGLT distribution and function in mammals, including humans, non-human primates, and rodents. The use of wild type, transgenic, and/or knockout rodents can be particularly useful, e.g. SGLT2−/− mice, as is the use of patients with genetic disorders of SGLT1 (GGM) or SGLT2 (FRG).

Nonlimiting examples of radiographic techniques include PET, (including mini-PET and micro-PET), SPECT, and the like.

In a variation of this aspect of the invention, the method of monitoring sodium/glucose co-transporter activity in a mammal, in vivo, comprises administering to a mammal a bolus of a tracer known to bind to sodium/glucose co-transporter 2 (SGLT2), but not sodium/glucose cotransporter 1 (SGLT); generating radiographic data indicative of tracer uptake in the mammal by scanning the mammal using a radiographic technique; and using the radiographic data to assess SGLT2 distribution or activity in the mammal. Advantageously, the radiographic technique can include, or be used in conjunction with, a computerized tomographic (CT) technique to scan all or part of the mammal's body, thereby providing an anatomical determination of the test animal and, hence, quantitation of tracer uptake into tissues and organs, both in the presence and in the absence of one or more pharmacological or other agents.

Optionally, additional information can be obtained by also administering one or more pharmacological or other agents to the animal or human subject, and monitoring the effect of the agent(s) on tracer uptake and distribution. Representative examples of suitable agents include phlorizin and other drugs in clinical trials (see FIG. 1 and Table 1). Phlorizin is a non-toxic compound and is a competitive, non-transported blocker of sugar transport by SGLT1 and 2 (Ki <1 μM), whereas other agents are high affinity, selective competitive inhibitors of SGLT1 or 2 (e.g. Table 1 FIG. 1). One skilled in the art based on the teachings herein and particularly FIG. 3 and representative compounds listed below will recognize that other compounds are suitable for the use as described herein.

In one embodiment of the invention, the number of SGLTs is determined by pharmacological experiments. Nonlimiting examples include: (1) intravenous injection of the specific SGLT blocker phlorizin, and (2) intravenous infusion of insulin and other anti-diabetic drugs.

It has been found that imaging methods and analytic methods currently practiced for GLUTs (using 2-FDG) are readily utilized with the new tracers described herein to assess SGLT distribution and activity. The following references describe PET, micro-PET, and autoradiographic techniques that are useful in practicing the invention: (1) Phelps M. E. PET Molecular imaging and its biological applications Springer, N.Y., 2004, including Chapter 1. Cherry, S. R. & Dahlborn, M. PET Physics, Instrumentation and Scanners; Chapter 2 Gambhir, S. S. Quantitative Assay Development for PET; Chapter 4. Barrio, J. R. The molecular basis of disease; Chapter 5, Czernin, J. Oncological applications of FDG-PET; and Chapter 7. Silverman D. H. S. & Melega, W. P. Molecular imaging of biological process with PET. (2) Moore T H, et al. Quantitative assessment of longitudinal metabolic changes in after traumatic brain injury in the adult rat using FDG-microPET. J Cereb Blood Flow Metab. 20(10):1492-501, 2000; (3) Matsumura A, et al. Assessment of microPET performance in analyzing the rat brain under different types of anesthesia: comparison between quantitative data obtained with microPET and ex vivo autoradiography. Neuroimage 20: 2040-2050, 2003; and (4)(Huang, Truong et al. 2005).

The kinetics of tracer uptake is obtained by tracer kinetic modeling (see for example Carson R. E. Tracer Kinetic Modeling. In: Valk P. E. et al. Positron Emission Tomography, Springer, 2003, and Gambhir, S. S. Quantitative Assay Development for PET. In: Phelps M. E. PET Molecular imaging and its biological applications Springer, N.Y., 2004).

Quantitative comparisons of uptakes observed with patients and with normal control subjects provide information about pathologies. For example, in those tumors that use SGLTs to obtain glucose as a fuel, these SGLT tracers can be used to stage the tumor and to monitor the effectiveness of surgery, and chemo- and/or radiation therapy. The methodology is similar to that used for the diagnosis; staging, restaging and monitoring of tumors that accumulate 2-FDG (see Czernin, J. Oncological applications of 2-FDG PET. In: PET, Molecular Imaging and its Biological Applications, Ed: Phelps, M. E. Springer, N.Y. 2004). It is noted that many tumors that consume glucose do not take up 2-FDG.

Oral administration of the molecular imaging probes will provide information about their time-dependent tissue accumulation and biodistribution.

Use of the tracers for in vivo and in vitro monitoring of therapeutic interventions of drugs on SGLTs, allows researchers to evaluate the site of action, dose dependency, length of action and other pharmacokinetic and pharmacodynamic parameters of the drug in animals or human subjects. For example, in one embodiment of the invention, a radiolabeled tracer as described herein is administered to a subject, and PET imaging is used to monitor the effects of drugs on the absorption of tracer from the gut, the reabsorption of tracer from the glomerular filtrate and the uptake of tracer into organs, tissues and tumors. The imaging studies can be carried out before, during and after drug administration. Such drug studies include those designed to promote glucose excretion by the kidneys, block glucose uptake into tumors, and chemotherapeutics.

These compounds and procedures described herein enable one to follow in real time the metabolism and elimination of SGLT drug metabolites by the liver and/or kidney (e.g., by using a specific radiolabeled drug). This permits the exploitation of the data to design modified drugs to reduce or eliminate undesirable metabolism for increased efficacy, e.g. reduce glucuronidation of SGLT2 inhibitors by pharmacological methods and/or chemical modification of the SGLT2 inhibitor.

18F]-Dapagliflozin and related molecules are also particularly useful diagnostic probes for the following:
(1) Disorders of glucose handling in diabetics and other metabolic disorders.
(2) In the kidney evaluation of patients with renal failure and patients susceptible to gout.
(3) Heart failure and related cardiovascular diseases.
(4) Various forms of cancer, including for monitoring of targeted therapies.
(5) Brain disorders such as stroke, ataxias, and Alzheimers.

### REFERENCES

Chao, E. C. and R. R. Henry (2010). “SGLT2 inhibition—a novel strategy for diabetes treatment.” Nat Rev Drug Discov 9(7): 551-559.
Helmke, B. M., C. Reisser, et al. (2004). “Expression of SGLT-1 in preneoplastic and neoplastic lesions of the head and neck.” Oral Oncol 40(1): 28-35.
Huang, S. C., D. Truong, et al. (2005). “An internet-based “kinetic imaging system” (KIS) for MicroPET.” Mol Imaging Biol 7(5): 330-41.
Hummel, C., Lu, C., Loo, D D F., Hirayama, B A., Voss, A A., and Wright, E M. (2010). “Glucose Transport by Human Sodium Glucose Co-Transporters. Am. J Physiology Cell Physiol. 300: C14-C21. 2011 (first published online Oct. 27, 2010).
Ishikawa, N., T. Oguri, et al. (2001). “SGLT gene expression in primary lung cancers and their metastatic lesions.” Jpn J Cancer Res 92(8): 874-9.
Komoroski, B., N. Vachharajani, et al. (2009). “Dapagliflozin, a novel, selective SGLT2 inhibitor, improved glycemic control over 2 weeks in patients with type 2 diabetes mellitus.” Clin Pharmacol Ther 85(5): 513-9.
List, J. F., V. Woo, et al. (2009). “Sodium-glucose cotransport inhibition with dapagliflozin in type 2 diabetes.” Diabetes Care 32(4): 650-7.
Oku, A., K. Ueta, et al. (1999). “T-1095, an inhibitor of renal Na+-glucose cotransporters, may provide a novel approach to treating diabetes.” Diabetes 48(9): 1794-800.
Scholl-Burgi, S., R. Santer, et al. (2004). “Long-term outcome of renal glucosuria type 0: the original patient and his natural history.” Nephrol Dial Transplant 19(9): 2394-6.
Washburn, W. N. (2009). “Development of the renal glucose reabsorption inhibitors: a new mechanism for the pharmacotherapy of diabetes mellitus type 2.” J Med Chem 52(7): 1785-94.
Wright, E. M., B. A. Hirayama, et al. (2007). “Active sugar transport in health and disease.” J Intern Med 261(1): 32-43.
Wright, E. M., Loo, Donald D. F., Hirayama, Bruce A. (2011). “Biology of Human

Sodium Glucose Transporters.” Physiol Rev 91:733-794.
Wright, E. M. and E. Turk (2004). “The sodium/glucose cotransport family SLC5.” Pflugers Arch 447(5): 510-8.

1. A radiolabeled sodium/glucose cotransporter inhibitor comprising a six membered sugar ring connected to a Ring A which is in turn connected to a Ring B wherein the inhibitor is a compound of the formula: [chemical structure]
Where X═—O—, —S—, —C—, CH₂, C—(H)alkyl, C(alkyl)₂), —NH— or N-alkyl,
1A, 1B, 2A, 2B, 3A and 3B═—H, —OH, —O—, alkyl, —F, —¹⁸F, —I, —¹²³I, —¹²⁴I or Z connected to the Ring A and Ring B moiety)
4A and 4B═—H, —OH, —F, ¹⁸F, —I, —¹²³I, —¹²⁴I, or Z connected to the Ring A and Ring B moiety, and
5A and 5B═—H, —OH, —CH₂OH, —F, ¹⁸F, —I, ¹²³I, —¹²⁴I—CH₂, —CH₂¹⁸F, —CH₂I, CH₂¹²³I, CH₂¹²⁴I, Z or CH₂Z, where Z is connected to the Ring A and Ring B moiety, and D- and L- isomers thereof,
Where Ring A and Ring B are selected from:
Phenyl rings,
Heterocyclic rings which can be a pyrrole, imidazole, thioimidazole, pyridine, furane, oxazole, pyrimidine or pyrolidine rings, or
Fused aromatic rings which can be a naphthalene, benzothiazole, benzopyrazole, quinoline, benzoxazole or indole rings,
Where the R substitutions in Ring A and Ring B are one or more halogens selected from the group consisting of F, Br, I or ¹⁸F, ¹²³I, ¹²⁴I, ⁷⁵Br or an alkyl, alkoxy, alkylamine, alkylthio, aryl, or a heterocyclic ring,
Where the Y link is —CH₂, alkyl substituted CH₂, —NH, N-alkyl, —O—, —S—, or
Where the Z link comprises either
Ring A attached directly to C₁, C₂, C₃, C₄, C₅ or C₅CH₂— in the six member sugar ring ,
or
Ring A attached to C₁, C₂, C₃, C₄, C₅, C₅CH₂— via Z where is Z is CH₂, or an alkyl substituted CH₂, —NH, N-alkyl, —O—, or —S—.

2. A method of in vivo assessment of sodium/glucose cotransporter (SGLT) distribution or activity in a mammal, wherein said mammal is selected from the group consisting of humans, non-human primates, rodents, wild-type rodents, transgenic rodents and knockout rodents, comprising administering to said mammal one or more compounds set forth in claim 1.

3. The method of claim 2 wherein said mammal includes non-human and human subjects with disturbances in glucose homeostasis.

4. The method of claim 3 wherein said disturbances in glucose homeostasis includes Type 1 and Type 2 diabetes, cancer, heart disease, gout, and brain disorders.

5. The method of claim 2, wherein the assessment comprises the use of radiographic techniques.

6. The method of claim 5 wherein the radiographic techniques comprises positron emission tomography (PET), micro-PET, mini-PET, or single-photon emission computerized tomography (SPECT)or radioautoradiography.

7. The method of claim 2 wherein one or more of the compounds set forth in claim 1 are administered to the mammal by oral delivery or injection, along with at least one agent selected from the group consisting of sodium ion, phlorizin, glucose, galactose, SGLT inhibitors and a drug.

8. The method of claim 7 wherein the drug is insulin.

9. A method of detecting a sodium/glucose cotransporter (SGLT) in vitro, comprising:
forming a compound as set forth in claim 1, exposing one or more single mammalian cells, a mammalian tissue sample or a mixtures thereof to said compound to form radiolabeled cells or tissue, and assessing the radioactivity of said radiolabeled cells or tissue.

10. A method of monitoring sodium/glucose cotransporter activity in a mammal, in vivo, comprising: administering to a mammal a bolus of a tracer for SGLT activity, and a compound which is an inhibitor for sodium/glucose cotransporter 2 (SGLT2), but not an inhibitor of sodium/glucose cotransporter 1 (SGLT1); generating radiographic data indicative of tracer uptake in the mammal by scanning the mammal using a radiographic technique; and, using the radiographic data to assess the distribution of SGLT1 and SLGT2 in the mammal, the radiographic technique comprising one or more computerized methods.

11. A method of preparing the compounds of claim 1 comprising reacting a sugar with an aryl compound in accordance with the procedure set forth in FIG. 7.

12. Synthesis of radiolabeled, [¹⁸F]- dapagliflozin ((1S)-1,5-anhydro-1-C-{4-chloro-3[(4-ethoxyphenyl)methyl]phenyl}-[18F]-4-deoxy-D-glucitol) comprising the procedure as set forth in FIG. 4.

13. A sodium/glucose cotransporter 2 inhibitor for use in the treatment of diabetes and other related disorders and a radiolabeled sodium/glucose cotransporter 2 inhibitor for imaging as set forth in claim 1 comprising 3-O-alkyl or 3 deoxy-D-glucose derivatives, said derivatives having a low in vivo metabolism and low rate of glucuronidation.
